(19) United States
(12) Patent Application Publication
(10) Pub. No.: US 2024/0410882 A1
(43) Pub. Date: Dec. 12, 2024

(54) DUAL-MODE SENSOR FOR BREAST CANCER CELL DETECTION

(71) Applicant: Univ. Electronic Science and Technology of China, Chengdu (CN)

(72) Inventors: Hua FAN, Chengdu (CN); Tianchi YANG, Chengdu (CN); Wei ZHANG, Chengdu (CN); Jing LUO, Chengdu (CN); Xinkai GUO, Dongguan City (CN); Yan SUN, Dongguan City (CN)

(21) Appl. No.: 18/458,128

(22) Filed: Aug. 29, 2023

(30) Foreign Application Priority Data
Jun. 12, 2023 (CN) 202310693299.2

(51) Int. Cl. G01N 33/543

(52) U.S. Cl. CPC G01N 33/54326; G01N 2800/7028

(57) ABSTRACT

A dual-mode sensor structure applied to the detection of breast cancer cells in blood samples is disclosed, along with a method of using the dual-mode sensor to detect magnetic particles coupled with antibodies in blood. The sensor generates a magnetic field by electrification of a wire or coil to attract the magnetic particles to the surface of the sensor. The sensor includes a small-sized TMR sensor and Hall sensor array equivalent in size to magnetic particles, ensuring that the sensor can generate a significant output voltage when particles are present. A baffle in the TMR sensor can fix the particles, separating each particle to prevent particle concentration or multiple particles from accumulating along the depth of the sensor without being accurately detected. This improves the linearity and accuracy of the sensor output voltage and detection of the number of magnetic particles. The sensor can detect the number of magnetic particles quickly. Moreover, by combining two different sensor modes, the sensor is versatile and can switch working modes according to different applications and/or environments.

### RELATED APPLICATIONS

The present application claims priority to Chinese Pat, application No. 202310693299.2, filed Jun. 12, 2023, the contents of which are incorporated by reference herein in their entirety.

### TECHNICAL FIELD

The present invention belongs to the fields of breast cancer marker detection, magnetism, microelectronics and solid electronics. The traditional magnetic particle detection has two defects. Traditional Tunnel Magneto Resistance (TMR) sensors detect magnetic particles on the sensor surface by electrification of wires, but do not consider the three-dimensional accumulation of magnetic particles in grooves, which affects the accuracy and linearity of the sensor. The invention concerns a sensor and a method of using the sensor, based on a dual mode or combination of a Hall sensor and a TMR magnetic sensor to detect magnetic particles combined with breast cancer markers. Using different sizes of TMR and Hall sensors, different working modes can be employed to detect magnetic particles of different sizes, and the groove structure of embodiments of the invention may optimize the linearity of the TMR sensor compared with the traditional structure.

### BACKGROUND

### (1) Breast Cancer Cell Detection

Breast cancer is one of the main causes of female deaths worldwide, so early diagnosis and monitoring of cancer is crucial. Epidermal growth factor receptor-2 (HER2) is one of the more thoroughly studied breast cancer genes so far, which is largely expressed in SK-BR-3 cells. At present, it is possible to combine anti HER2 antibodies with magnetic nanoparticles through antibody coupling technology, and then combine anti HER2 antibodies with HER2 antigen, so as to achieve the purpose of separating SK-BR-3 cancer cells from human whole blood. For example, relevant literature may include Hengyi Xu, Zoraida P. Aguilar, Lily Yang, Min Kuang, Hongwei Duan, Yonghua Xiong, Hua Wei, Andrew Wang, Anti body combined magnetic iron oxide nanoparticles for cancer cell separation in fresh whole blood, Biomaterials, Volume 32, Issue 36, 2011, Pages 9758-9765, ISSN 0142-9612. FIG. 1 is a schematic diagram of the preparation of antibody coupled MNPs Si.

### (2) Magnetics

When a substance is subjected to an external magnetic field B0, it will be magnetized, generating an additional magnetic field B′, resulting in a total magnetic field B=B0+B′. For example, a coil with an iron core will generate a larger magnetic field because B′ is generally much larger than B0. According to the different values of B′, substances can be simply divided into three categories: paramagnetic materials, diamagnetic materials, and ferromagnetic materials. The B′ generated by both paramagnetic and diamagnetic materials is particularly small and is generally not considered.

### (3) Microelectronics and Solid State Electronics

Tunnel magnetoresistance sensors' ultra-high sensitivity and reliability have made them widely studied and applicable in military and civilian fields. The structure of a typical tunnel magnetoresistance sensor includes, from top to bottom, a free layer, a tunnel barrier, a pinning layer, and an antiferromagnetic layer.

In a multilayer TMR sensor structure composed of ferromagnetic layer/barrier layer/ferromagnetic layer, because the Fermi surface densities of layers containing many Spinons versus a few Spinons are different, the probabilities of carrier tunneling in the parallel state versus the antiparallel state are also different, and the different tunneling probabilities result in different resistance values of the resulting magnetic tunneling structures. The change in resistance value is approximately linearly related to the magnitude of the external magnetic field.

Due to the fact that the magnetic field is a vector field, the change in resistance value in the magnetic tunneling junction is related to the angle between the magnetization direction, as shown in FIG. 2. If the internal magnetic field of the tunneling magnetoresistance sensor is Bi and the external environmental magnetic field is Bo, when the external magnetic field Bo changes, the direction and magnitude of the total magnetic field B detected by the tunneling magnetoresistance sensor will also change. If the angle between the total magnetic field B and the direction of the tunneling current is θ, according to the tunneling magnetoresistance effect, the change in θ will cause the magnetic tunneling junction resistance R(θ) to change.

The working principle of the TMR sensor is as follows: Applying current to a wire will generate a first magnetic field, which will act on the tunnel magnetoresistance sensor and magnetic nanoparticles. In this first magnetic field, the magnetic nanoparticles are magnetized, and the magnetized nanoparticles will generate a second magnetic field, which can be called a stray field. The stray field and the sensor's own magnetic field combine to generate a third magnetic field. These different magnetic fields cause changes in the sensor resistance. Ultimately, it is reflected in changes in voltage.

Hall sensors are typical magnetic sensors based on the Hall effect, and are widely used in measuring displacement, angle, velocity, current, and voltage (e.g., of or induced by a magnetic field). The Hall effect was discovered by scientist Hall in 1879 while studying the conductive mechanism of metals. As shown in FIG. 3, when a current (or voltage) passes through a semiconductor perpendicular to an external magnetic field, the carrier deflects due to the Lorentz force. An additional electric field is generated perpendicular to the direction of the current and magnetic field, resulting in a potential difference at both ends of the semiconductor. This potential difference is called the Hall voltage. Later, it was discovered that materials such as semiconductors and conductive fluids also exhibit this effect, and the Hall effect of semiconductors is much stronger than that of metals. Additionally, Hall sensors have been widely used due to the compatibility of Hall sensors with mainstream manufacturing processes, such as complementary metal oxide semiconductor (CMOS) processes.

### (4) Reconfigurable Multimodal Sensor Interface Circuit

The vast majority of analog front-end circuits are designed for a single type of target sensor and have unique functions and applications. Developing interface circuits for specific sensors consumes a significant amount of time and cost, and its system flexibility and scalability are also greatly limited. The interface circuit of a specific target sensor is only suitable for large-scale, high-cost industrial production. In order to meet certain market demands, a reconfigurable analog front-end circuit suitable for general sensor platforms may be desirable. Sensors can convert non electrical signals such as physical, chemical, and biological signals into electrical or electrically-convertible forms of signals, which can be roughly divided into four types based on their output signal types: voltage (V), current (I), resistance (R), and capacitance (C). Reconfigurable analog front-end circuitry can accept different sensor output types and connection configurations, thus improving the efficiency of the circuit design, reducing costs and improving the flexibility of the system.

In order to achieve greater universality of sensors (for example, if one wants to detect the quantity of magnetic particles while also detecting the position(s) of the magnetic particles, or detect magnetic particles with different diameters), requirements for detection sensitivity may vary.

### (5) Cross Field

Compared with traditional optical biosensors, TMR sensors have attracted attention due to their lower background noise and better performance (see, for example, Su D, Wu K, Saha R, Peng C, Wang J P, “Advances in Magnetoresistive Biosensors,” Micromachines (Basel), 2019 Dec. 26; 11 (1): 34; Doi: 10.3390/mi11010034 PMID: 31888076; PMCID: PMC7019276).

In biomedicine, Hall sensors made of silicon materials have also been proven to detect the presence, quantity, and location of superparamagnetic (similar to ferromagnetic) particles with a diameter of 0.1-10 microns, as described in Gambini S, Skucha K, Liu P P, et al. A 10 kPixel CMOS Hall Sensor Array with Baseline Suppression and Parallel Readout for Immunoassays. IEEE Journal of Solid State Circuits, 2013, 48(1): 302-317.

At the same time, research on the binding of MNPS to cancer cells in biomedicine continues to deepen, as described in Amir Hossein Haghih, Zahra Faghih, Mohammad Taghi Khorasani, Fatemeh Farjadian, Antibody conjugated onto surface modified magnetic nanoparticles for separation of HER2+ breast cancer cells, Journal of Magnetism and Magnetic Materials, Volume 490, 2019, 165479, ISSN 0304-8853. Data measured using magnetic instruments indicate that magnetic nanoparticles can be effectively bound to cancer cells.

### SUMMARY OF THE INVENTION

The invention concerns a dual-mode sensor for detecting magnetic particles coupled to breast cancer markers in blood, a significant advance in the art in view of the fact that there is presently no chip for detecting breast cancer cells in blood.

The present invention is based on a technology that couples cancer cells to magnetic nanoparticles through antibodies, and then senses the number of magnetic nanoparticles and other information through a highly sensitive TMR magnetic sensor. Finally, we can detect breast cancer cells in blood. The basic idea of the present invention is to sense the presence and position of particles by measuring the enhanced magnetic field of iron oxide (e.g., iron oxide particles) through a magnetic sensor. In the present invention, Hall array sensors can be combined with TMR sensors using reconfigurable multimode sensor interface circuits to integrate multiple sensor functions on the same chip: it can quickly detect the number of magnetic particles through the resistance changes of TMR devices, and it is also possible to determine the position of the magnetic particles by analyzing voltage information from the Hall array sensors. Alternatively, the device size of the Hall sensor(s) can differ by an order of magnitude from the TMR sensor, so that when encountering particles of different sizes, more accurate detection can be achieved by switching the sensor's operating mode. All of these principles provide a basis for the detection of breast cancer cells in the blood using a dual-mode sensor.

The invention relates to a dual-mode sensor applied to the detection of breast cancer cells in blood samples, which comprises: a semiconductor substrate, a Hall sensor array, a metal coil and a TMR sensor. The Hall sensor array may be on one side of a semiconductor substrate, the TMR sensor may be on another side of the substrate, and the metal coil may be on one of the two sides of the semiconductor substrate and have a planar multi-layer ring structure (e.g., by winding). The Hall sensor array and the TMR sensor are in a center of the ring structure. The Hall sensor array (or one or more Hall sensors in the array) is not encapsulated.

The TMR sensor may comprise a TMR device layer, an isolation substrate, a wire layer and a silicon nitride adsorption layer. In a corresponding method of making the present sensor, the TMR device layer and the semiconductor substrate are bonded, and then the isolation substrate, the wire layer and the silicon nitride adsorption layer are formed thereon in order. There may be a bonding layer between the wire layer and the silicon nitride adsorption layer. The silicon nitride adsorption layer may include multiple adsorption tanks. Sides of the adsorption tanks may have a slope toward the adsorption tank. The wire may be above the TMR device, and the adsorption tank may be above the wire.

Antibodies (e.g., to breast cancer cells) bound to ferric oxide particles also bind to breast cancer cells (e.g., in the blood of a patient), and then the dual-mode sensor is used to detect the ferric oxide particles to achieve breast cancer cell detection.

Further, the adsorption tanks may include a silicon nitride baffle plate configured to form a series of silicon nitride adsorption cells.

The invention combines a Hall sensor and a TMR sensor to form a dual-mode magnetic sensor. In particular, the structure of the TMR sensor includes an adsorption layer that improves the detection accuracy and sensitivity of the dual-mode sensor.

### DESCRIPTION OF THE FIGURES

FIG. 1 is a manufacturing process diagram of MNPs having a silane coating thereon with anti-HER2 antibodies bound thereto;

FIG. 2 is a schematic diagram of a TMR sensor;

FIG. 3 is a schematic diagram of a Hall magnetic sensor;

FIG. 4 is a diagram showing an overall structure of a dual mode sensor;

FIG. 5 is a circuit structure diagram of an interface for the dual mode sensor configured to detect breast cancer cells;

FIGS. 6a-6j are TMR sensor simulation results and analysis diagrams; FIG. 6a is a schematic diagram of a stray magnetic field change on a TMR device caused by a single magnetic particle; FIG. 6b is a schematic diagram of the change of stray magnetic field on the TMR device caused by two magnetic particles; FIG. 6c is a schematic diagram of stray magnetic field changes on the TMR device caused by three magnetic particles; FIG. 6d is a schematic diagram of stray magnetic field changes on the TMR device caused by four magnetic particles; FIG. 6e is a schematic diagram of stray magnetic field changes on the TMR device caused by five magnetic particles; FIG. 6f is a perspective and plan view of stray magnetic field changes on an alternative TMR device caused by the three-dimensional accumulation of five magnetic particles without boards or baffles; FIG. 6g is cross-sectional views of stray magnetic field changes on the alternative TMR device caused by the three-dimensional accumulation of five magnetic particles without boards or baffles; FIG. 6h is a schematic diagram of stray magnetic field changes on the alternative TMR device caused by the three-dimensional accumulation of four magnetic particles without boards or baffles; FIG. 6i is a schematic diagram of stray magnetic field changes on the alternative TMR device caused by the three-dimensional accumulation of three magnetic particles without boards or baffles; FIG. 6j is a schematic diagram of stray magnetic field changes on the alternative TMR device caused by the three-dimensional accumulation of two magnetic particles without boards or baffles; FIG. 6k is a schematic diagram of stray magnetic field changes on the alternative TMR devices caused by three-dimensional accumulation of single magnetic particles without boards or baffles; FIG. 6l is a graph showing the resistance of the TMR device with and without boards or baffles; and FIG. 6m is a table of various simulation data for the TMR device with and without boards or baffles;

FIGS. 7a-p are voltage outputs of 64 Hall devices detecting changes in magnetic particles; and when magnetic particles are present, an additional magnetic field is generated nearby.

FIG. 8 is a comparison table of resistances of the TMR device with a barrier and without a barrier in the absorbing layer of the TMR sensor.

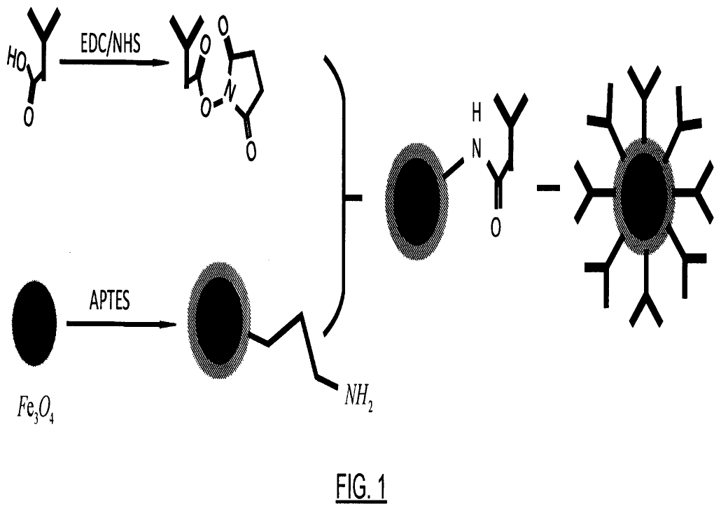

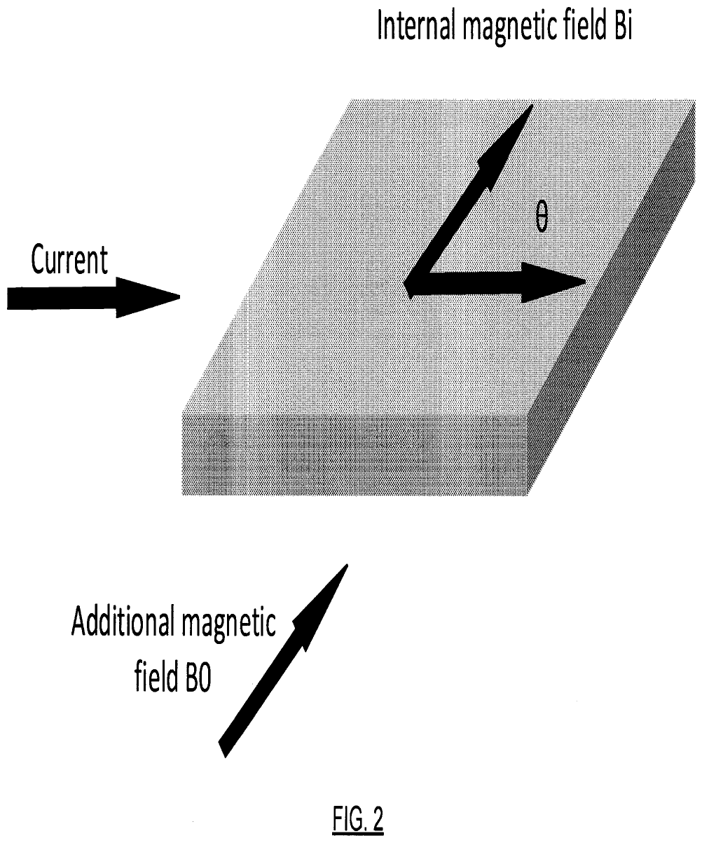

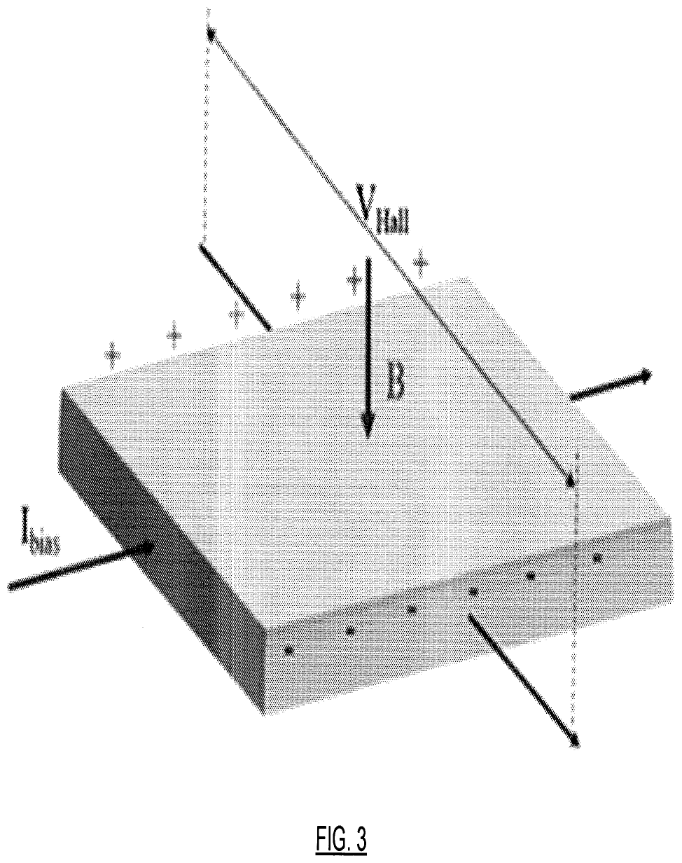

### DETAILED DESCRIPTION

The principle of detecting micron level particles using TMR sensors is as follows: an external magnetic field B0 is applied (e.g., by or to the sensor), and when particles are on the surface (e.g., of the sensor), the particles will magnetize to generate an additional magnetic field B′. The TMR sensor below or adjacent to the particles is used to capture changes in B′. The principle of Hall sensor detecting micron level particles is similar. Because particles in the blood are very small, the magnetic field area generated by magnetization is generally equivalent to the size of the particles. The metal particles in blood samples are mostly at the micron and submicron levels. Therefore, in order for the chip to sense the additional magnetic field B′ generated by the particles, the distance between the particles and the sensor should not be too large and should be controlled at the micron level. Usually, during the manufacturing process of chips, a layer of silicon nitride is deposited on the surface, typically with a thickness of a few micrometers. The thickness of the chip packaging is much greater than a few micrometers, so the chip should not be packaged or used in special packaging forms.

It is necessary to use Hall and TMR devices that are equivalent to the particle size, but one should ensure that the size of the Hall sensor is larger than that of the TMR sensor.

The reason for the equivalent size is that if the magnetic particles in the blood are too small, the device may experience little or no change in the sensor output voltage due to the perceived small magnetic field enhancement area when the particles generate a magnetic field enhancement area that is equivalent to their own size. If the magnetic particles in the blood are too large, it can lead to abnormal operation of the magnetic sensor, such as magnetic saturation of the TMR device or the voltage of the Hall sensor array reaching the maximum value, making it impossible to determine the number and position information of the magnetic particles.

The reason why Hall sensors have a larger size than TMR sensors is that TMR sensors are more sensitive than Hall sensors, so TMR sensors are used to detect smaller magnetic particles, and Hall sensors are used to detect larger magnetic particles. These two different sensors each perform their respective duties and achieve acceptable or optimal results within their respective detection ranges. At the same time, such design enhances the universality of magnetic sensors.

By applying electricity to the metal, magnetic force is generated to attract particles to the sensor surface.

To ensure stable and accurate measurement, it is necessary to attract oxide particles from the blood to the surface of the sensor. The electrification mainly follows the following two principles: the magnetic field generated by the coil (e.g., in the sensor) is perpendicular to the direction of the chip, placing the Hall sensor in a working state. The magnetic field generated by the wires of the TMR sensor is parallel to the direction of the chip, placing the TMR sensor in a working state. The generated magnetic field intensity must be greater than the geomagnetic field (30-70 μT), perhaps by up to one order of magnitude. This magnetic field can also be used for magnetization of surface particles. The additional magnetic field strength generated by magnetization of particles with high permeability in the blood can lead to changes in the resistance of the TMR device(s) and the Hall voltage of the Hall devices. Ultimately, it is converted into voltage values through a multi-mode interface circuit.

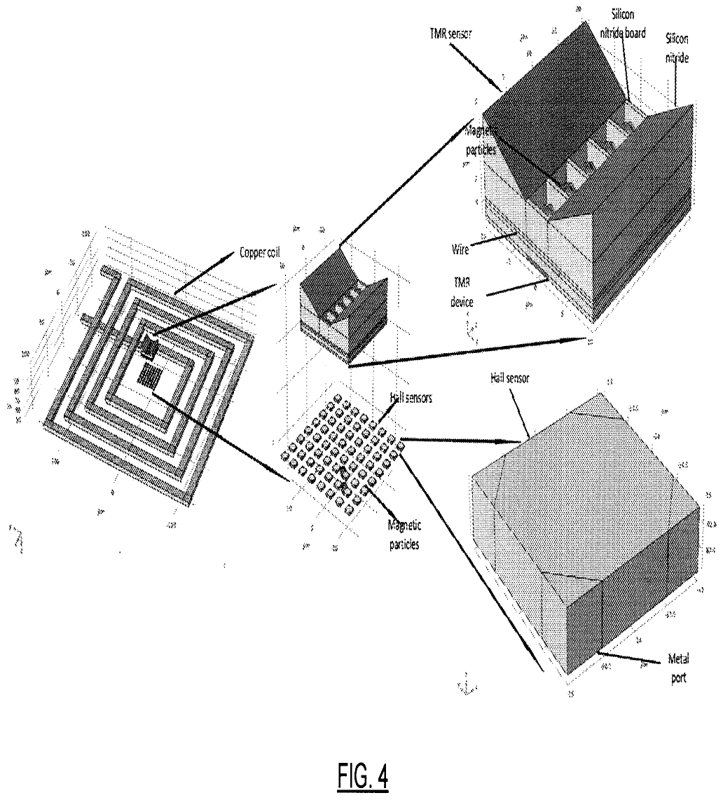

The TMR sensor and Hall magnetic sensing array are arranged back-to-back and/or in reverse as shown in FIG. 4. The purpose of the reverse arrangement is to ensure that the sensors can fully and directly contact magnetic particles in the liquid as close as possible in every operating state.

The TMR sensor is connected to the Hall sensing array through the interface circuit shown in FIG. 5, where the TMR sensor is connected to the resistance sensor of the circuit, and the Hall sensing array is connected to the voltage sensor of the circuit. The desired working mode (e.g., resistance detection or voltage detection) can be selected using the switch. The final output values are all voltages.

The invention concerns a dual-mode magnetic sensor which can quickly detect breast cancer cells in the blood, aiming to improve people's awareness of their health and prevent breast cancer or other related diseases.

The present invention establishes a physical model of the structure in FIG. 4 through the finite element simulation software COMSOL. The TMR sensor model selection included 4×20 micron rectangular TMR devices with a thickness of 0.4 microns, comprising a nonlinear magnetic material (e.g., nickel molybdenum steel), and powered by a current of 150 uA. The wire is a metal copper wire with a size of 8×20 microns, with a thickness of 0.4 microns, the wire is subjected to a current of 40 mA, generating a magnetic field of about 3 to about 3.2 mT on the surface. A protective layer with a distance of 1.5 microns between the TMR device(s) and the particles has a relative magnetic permeability of 10. When the present invention is in the TMR device operating mode, the finite element simulation software COMSOL can be used to calculate the changes in the resistance of the TMR device when different numbers of particles with a diameter of 1.2 micrometers are present.

The Hall device selected for the COMSOL model included a 2×2 micron square hall device with a thickness of 0.5 micrometers. Triangular areas at the four corners of the square were selected as metal to form ohmic contact with the semiconductor. The power supply voltage of a single Hall device was 5V. A single device is arranged 2 microns outside of another Hall device, resulting in the design of an 8×8 Hall device array. The coil is energized with a current of 30 mA, generating a magnetic field of 10⁻⁴ to 10⁻³ Tesla on the surface. The protective layer with a distance or thickness of 3 microns between the Hall device and the particles has a relative magnetic permeability of 10. When the present invention is in the working mode of a Hall device, the finite element simulation software COMSOL can be used to calculate the changes in Hall output voltage from the 64 Hall devices in the presence of particles with diameters of 2 microns and 1 micron.

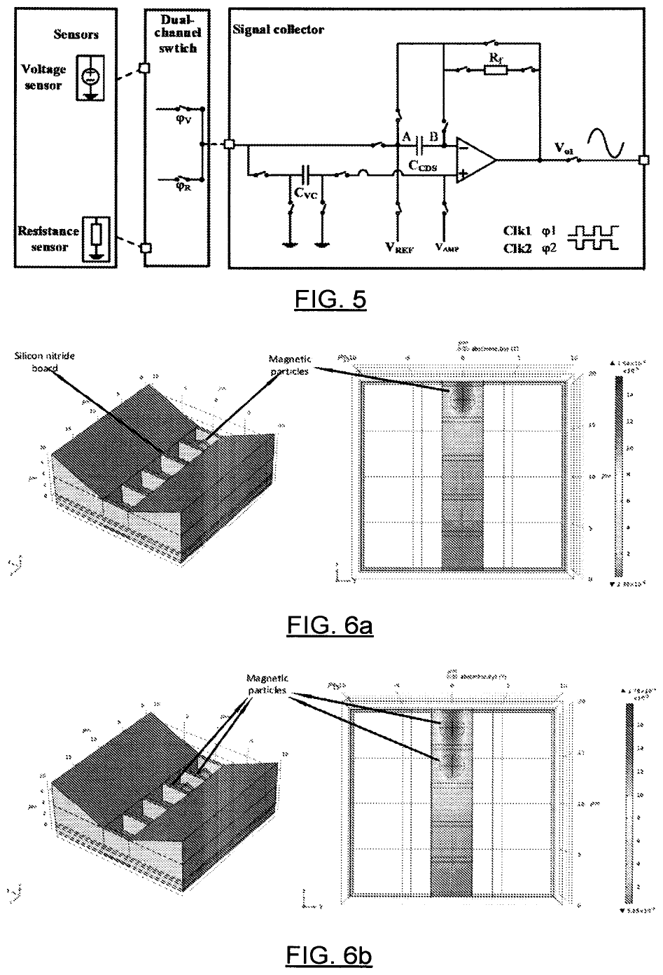

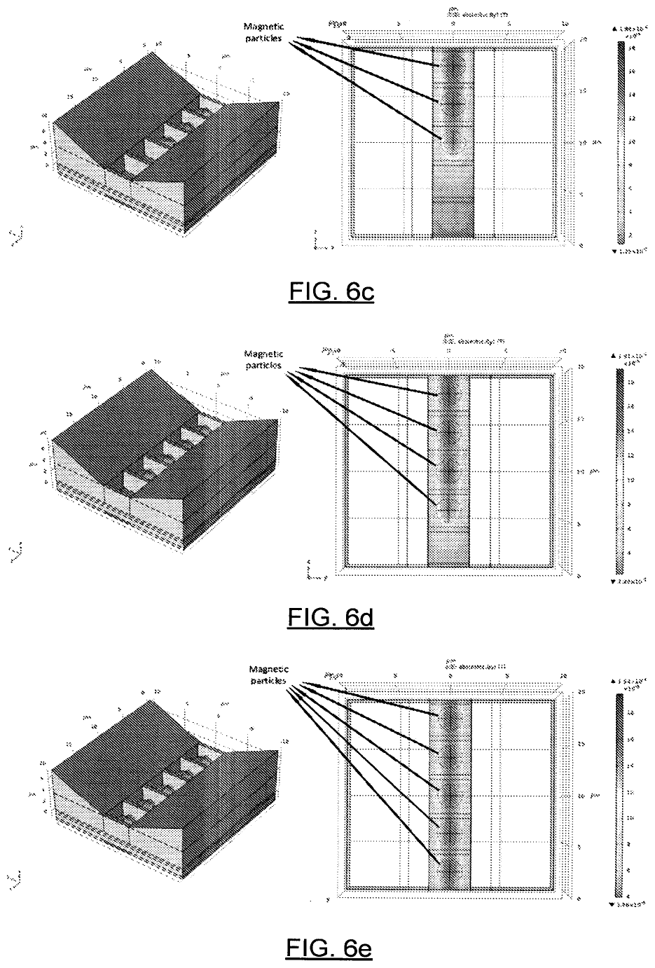

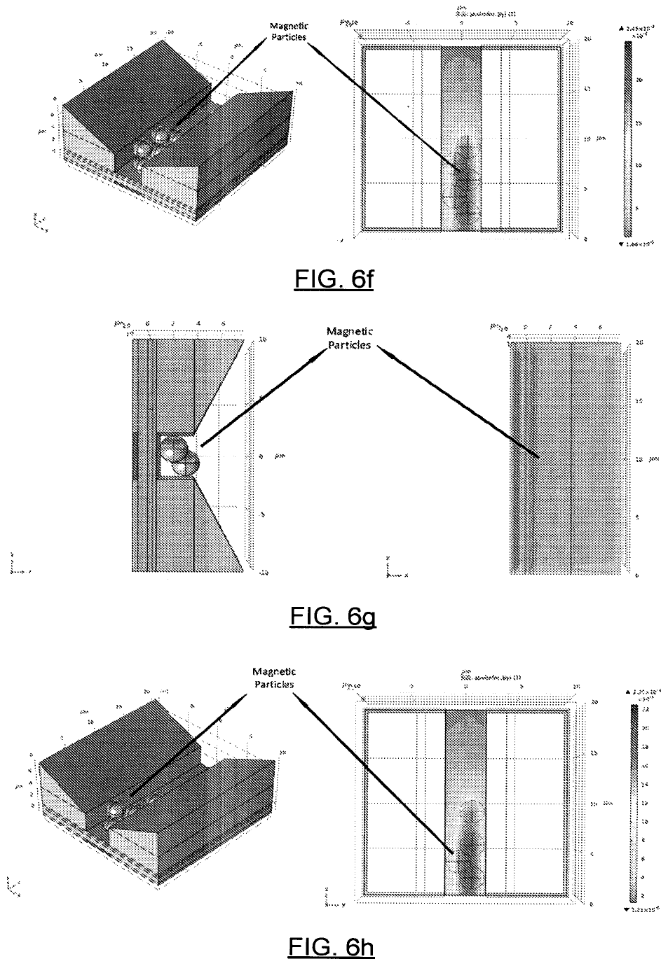

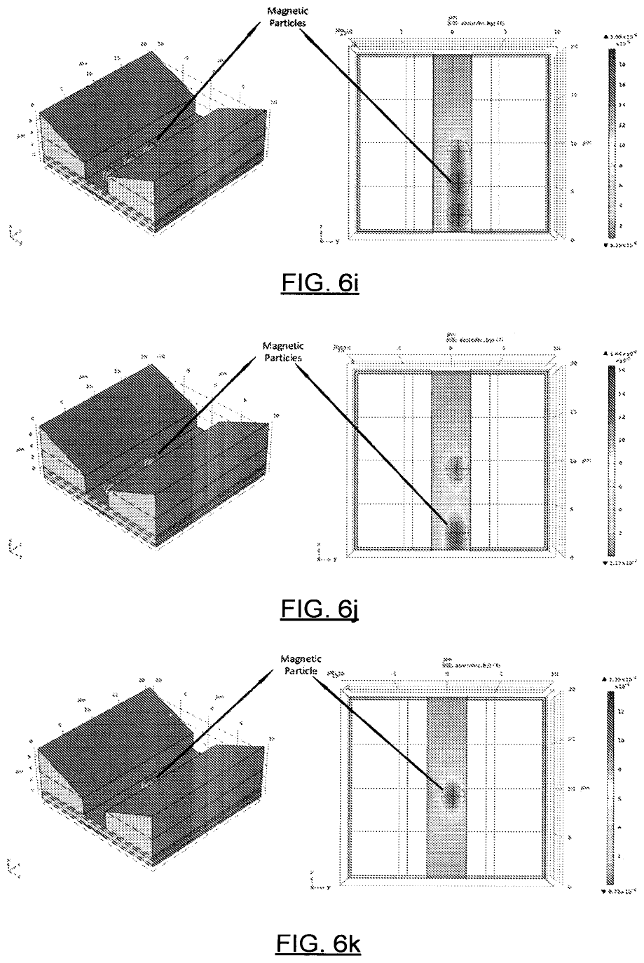

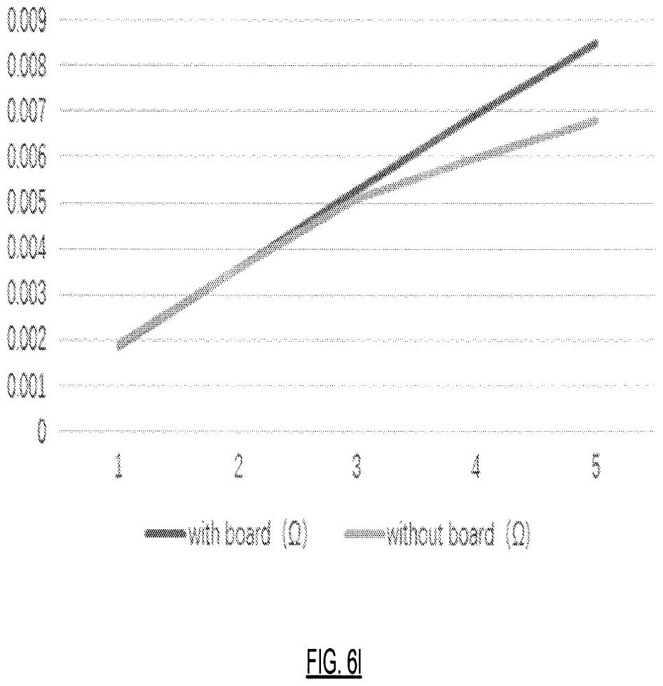

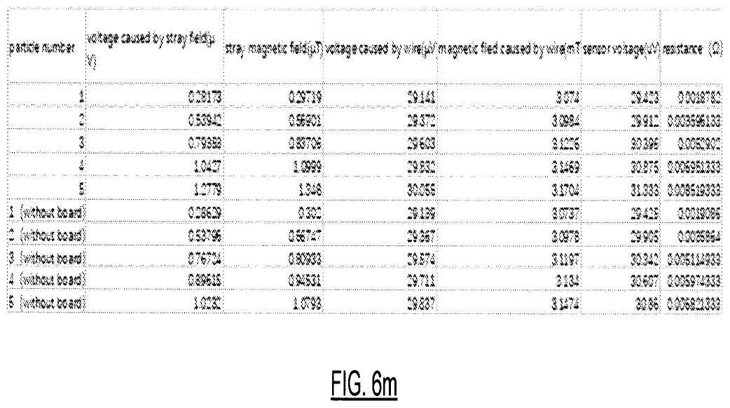

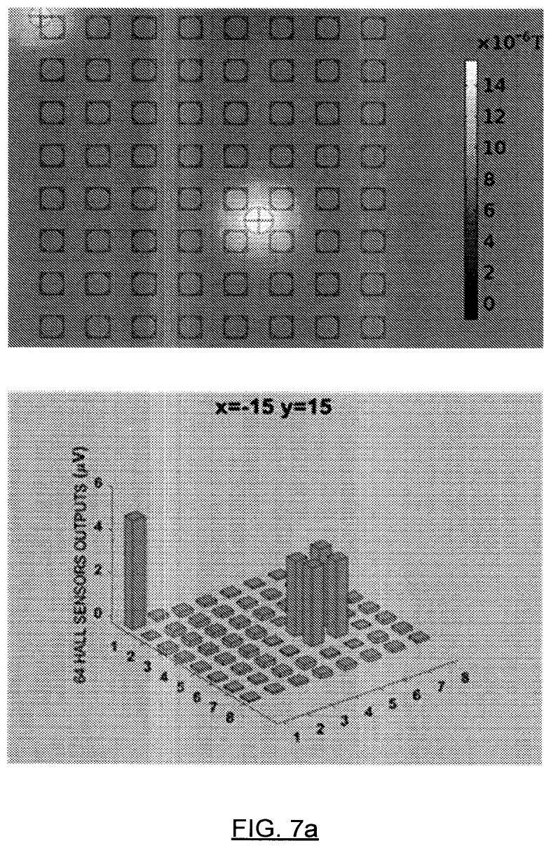

The numerical calculation results of the TMR sensor model are shown in FIGS. 6a-m, with a total of 12 images from FIG. 6a to FIG. 6k. The images are divided into two parts. The left part shows the location of magnetic particles in the sensor, while the right part shows the magnetization of the particles by the magnetic field generated by the wire, which in turn generates additional stray field intensity on the TMR sensor. The stray field intensity is about 30-130 micro Tesla. The table in FIG. 6m shows that when particles are present, the additional magnetic field generated by the particles will be perceived by nearby TMR devices. Simulation results show that when particles are present near a certain TMR sensor, there will be an output voltage change of around 0.2 to 1.2 microvolts and a corresponding magnetic field change. FIGS. 6a-e show the working condition of the sensor with a silicon nitride baffle. Magnetic particles are arranged linearly in the sensor, and the output voltage change generated also increases approximately linearly as shown in FIG. 6h. FIGS. 6f to 6k show that when the sensor does not have a silicon nitride baffle, the output results caused by the accumulation of magnetic particles in three-dimensional space are unpredictable, interfering with the accuracy of the sensor, It also leads to a decrease in sensor linearity. FIG. 6l shows the difference in linearity between TMR sensors with and without baffles. From FIG. 6l, it can also be seen that when the number of magnetic particles is small, the difference between the presence and absence of baffles is not significant. As the number of magnetic particles increases, the generated TMR resistance deviates from the linear relationship.

A table showing the data obtained from various simulation experiments is shown below:

| | voltage | | | magnetic | | |
|---|---|---|---|---|---|---|
| | caused | stray | voltage | filed | | |
| particle | by stray | magnetic | caused by | caused by | sensor | resistance |
| number | field(μV) | field(μT) | wire(μV) | wire(mT) | voltage(μV) | (Ω) |
| 1 | 0.28173 | 0.29719 | 29.141 | 3.074 | 29.423 | 0.0018782 |
| 2 | 0.53942 | 0.56901 | 29.372 | 3.0984 | 29.912 | 0.003596133 |
| 3 | 0.79353 | 0.83706 | 29.603 | 3.1226 | 30.396 | 0.0052902 |
| 4 | 1.0427 | 1.0999 | 29.832 | 3.1469 | 30.875 | 0.006951333 |
| 5 | 1.2779 | 1.348 | 30.055 | 3.1704 | 31.333 | 0.008519333 |
| 1 (without board) | 0.28629 | 0.302 | 29.139 | 3.0737 | 29.425 | 0.0019086 |
| 2 (without board) | 0.53796 | 0.56747 | 29.367 | 3.0978 | 29.905 | 0.0035864 |
| 3 (without board) | 0.76724 | 0.80933 | 29.574 | 3.1197 | 30.342 | 0.005114933 |
| 4 (without board) | 0.89615 | 0.94531 | 29.711 | 3.134 | 30.607 | 0.005974333 |
| 5 (without board) | 1.0232 | 1.0793 | 29.837 | 3.1474 | 30.86 | 0.006821333 |

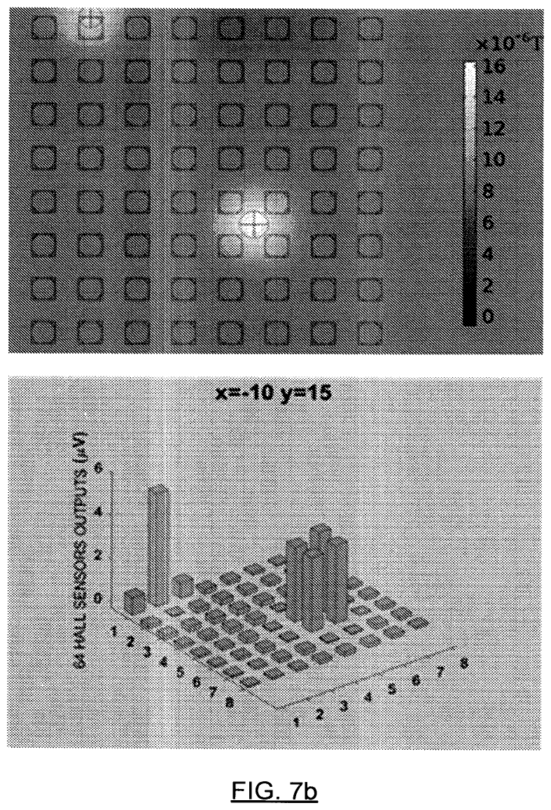

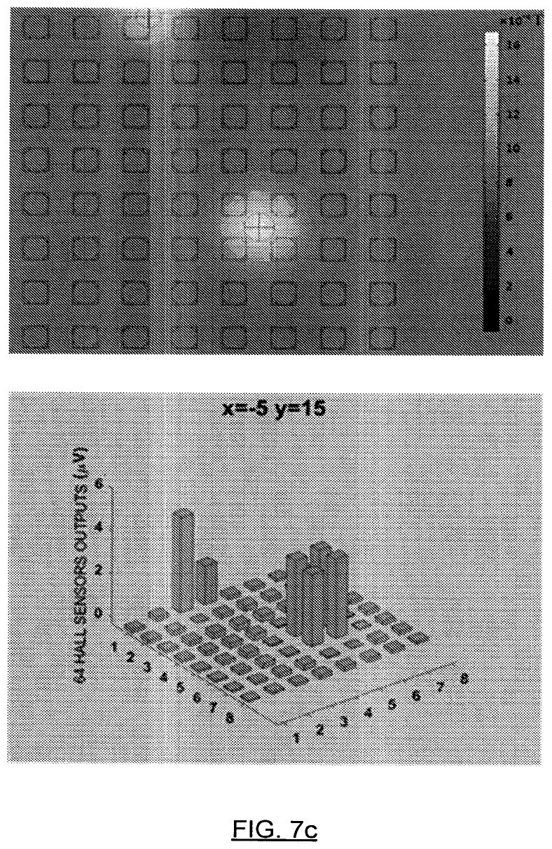

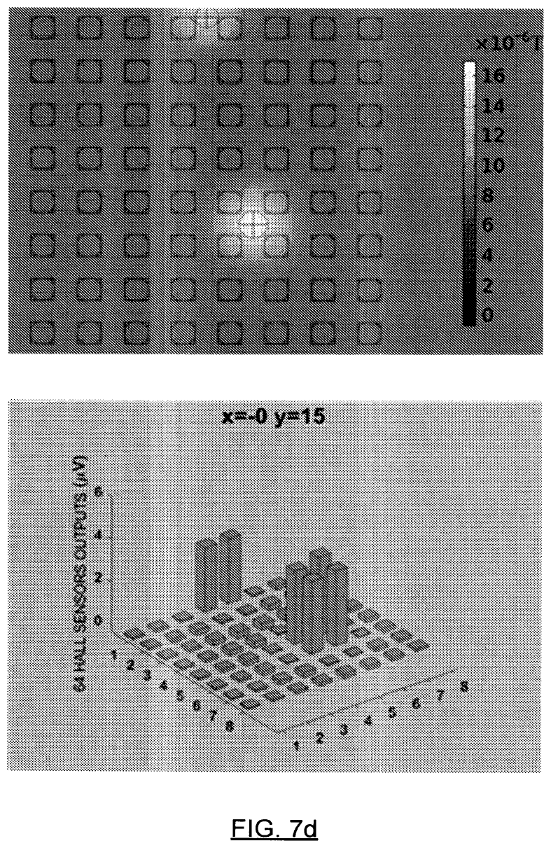

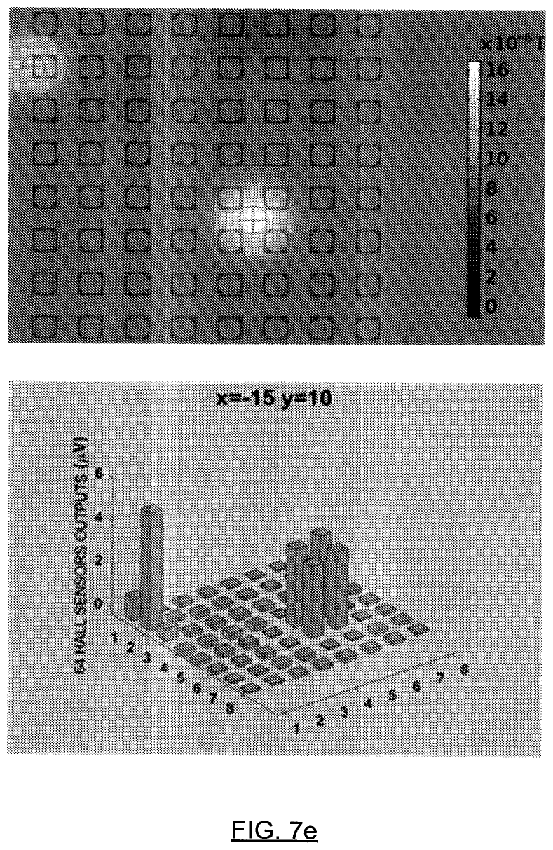

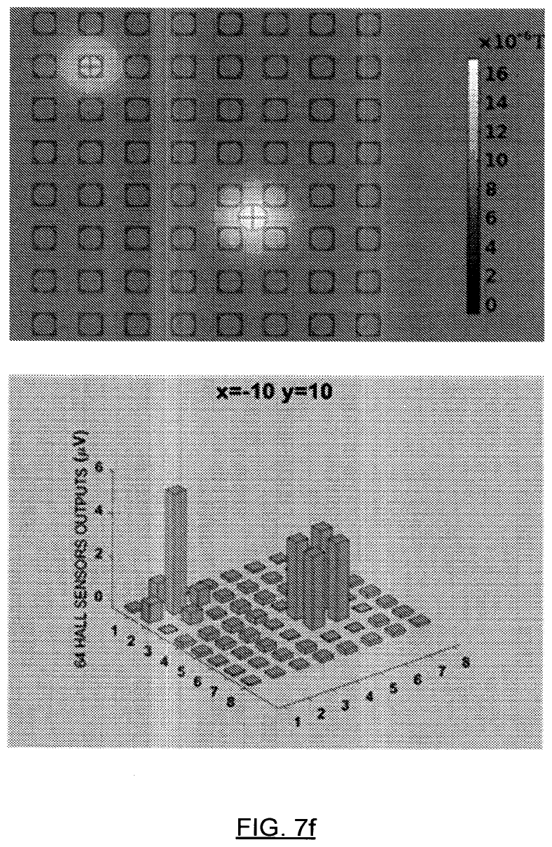

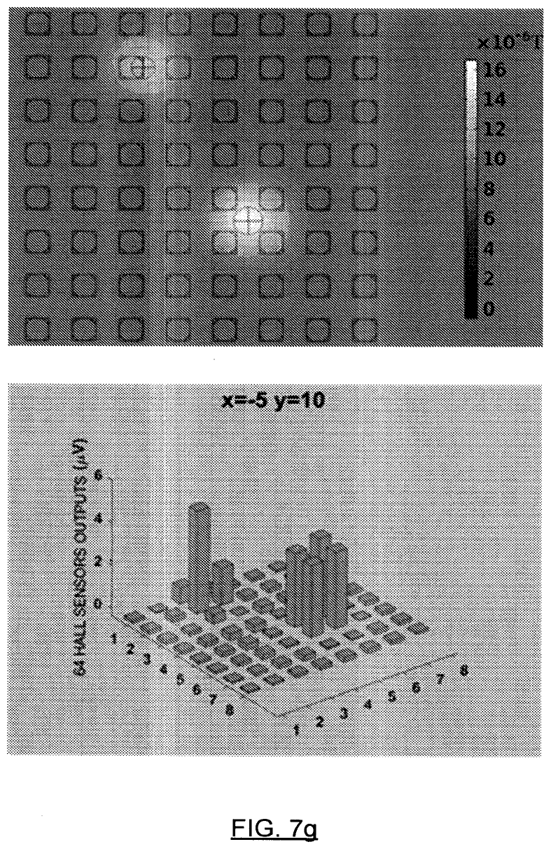

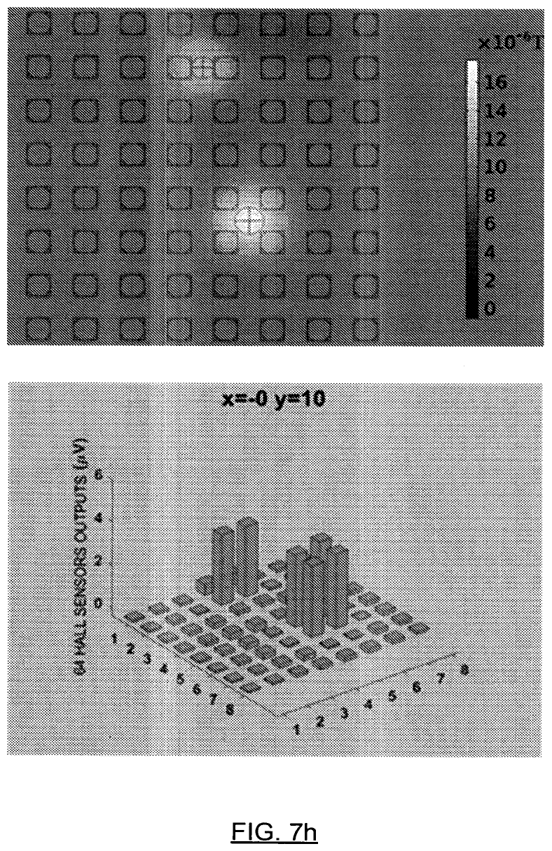

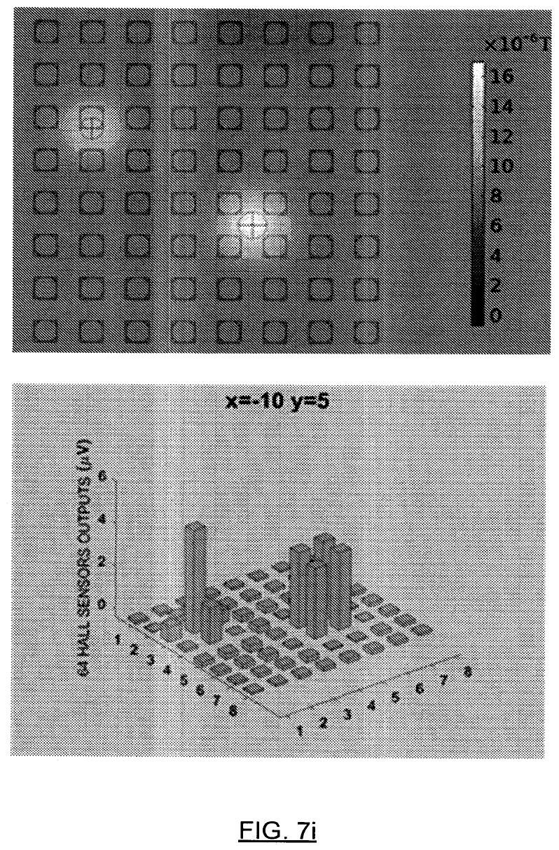

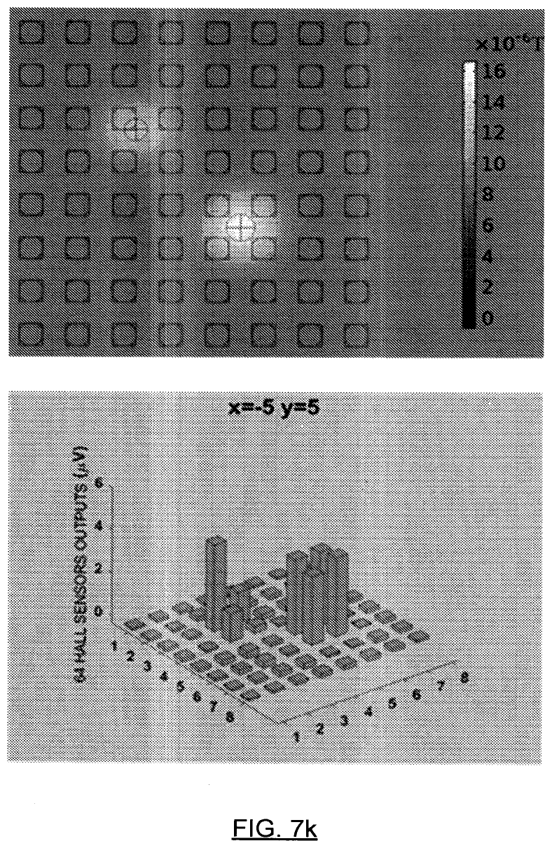

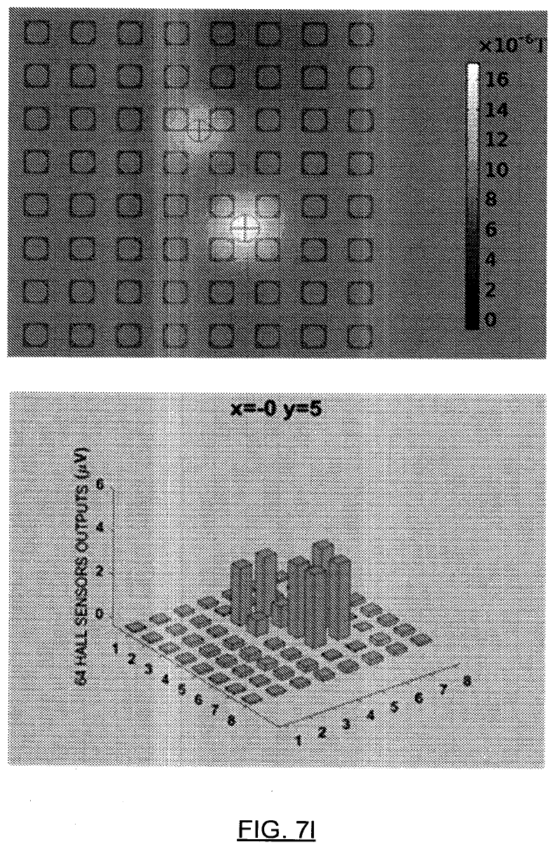

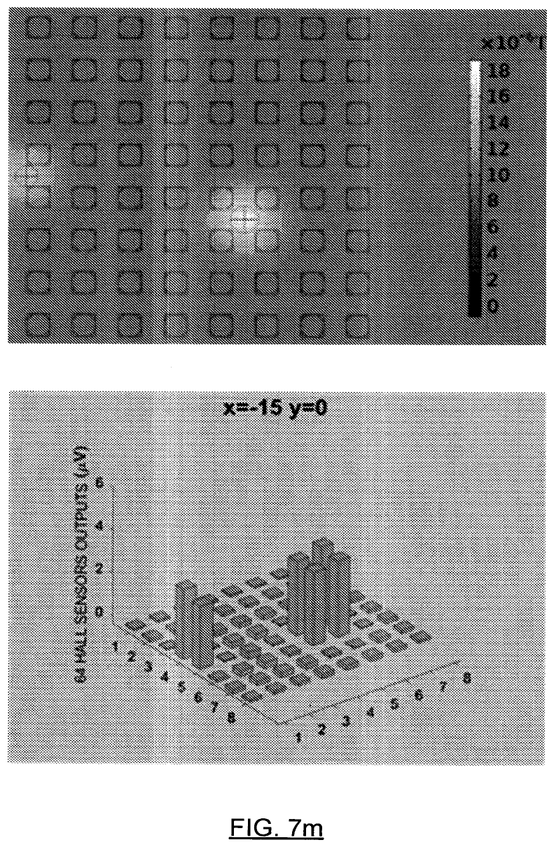

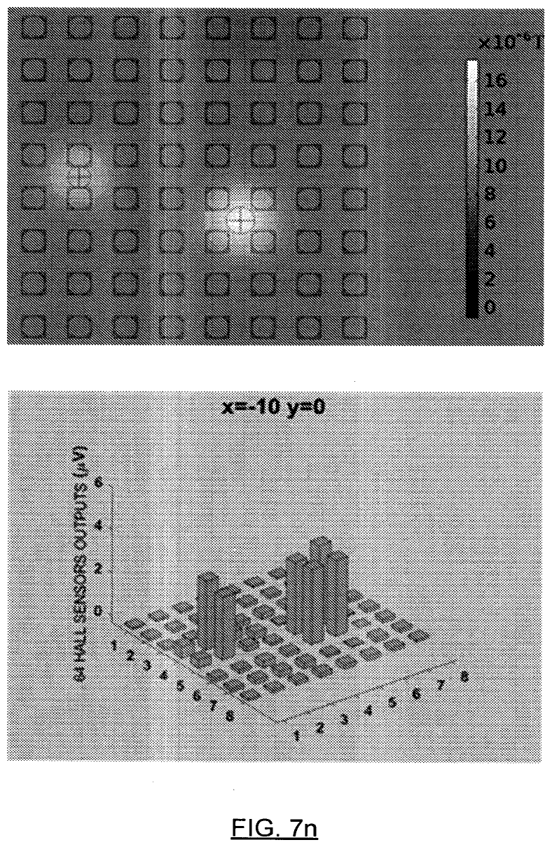

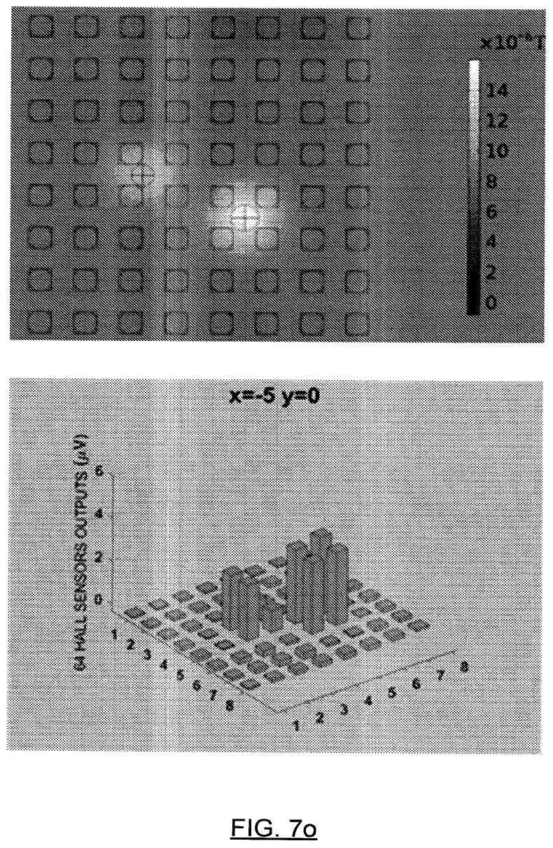

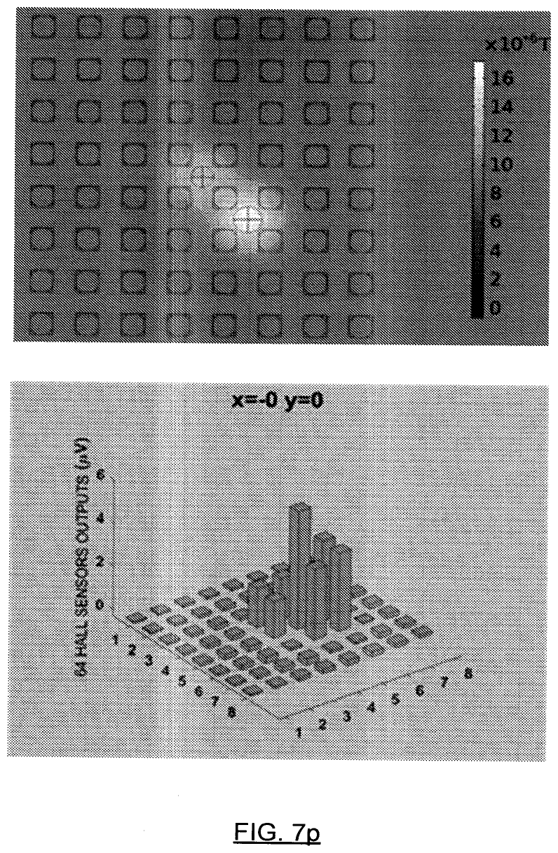

The numerical calculation results of the Hall magnetic array sensor model are shown in FIGS. 7a-7p, with a total of four images from FIG. 7a to FIG. 7d. Each image is divided into two parts. Firstly, when particles are present, additional magnetic fields will be generated nearby due to magnetization, with a magnetic field strength of approximately 1-20 micro Tesla; The second is that when particles are present, the additional magnetic field generated will be perceived by nearby Hall devices. Simulation results show that when particles are present near a Hall sensor, there will be an output voltage change of about 1-6 microvolts. In FIG. 7j, the changes in the Hall voltage output of 64 Hall devices were scanned from FIG. 7a to FIG. 7p when particles with a diameter of 1 micrometer were placed at different positions. In this simulation, 64 Hall devices were selected in the upper left corner, with a total of 16 points spaced at 5 microns intervals, resulting in 16 images (FIG. 7a to FIG. 7p). During this process, particles with a diameter of 2 microns remained stationary. In addition to significant voltage changes generated by nearby Hall sensors when particles are present in different positions, the signal changes of Hall devices also vary depending on the particle position. It can be roughly divided into: the center of the circle at the angle of the Hall device (FIGS. 7a, 7c, 7i, 7k); the center of the circle is at the center of one side of the Hall device (FIGS. 7b, 7e, 7g, 7j); the center of a circle on one side of two Hall devices (FIGS. 7d, 7l, 7m, 7o); the center of the circle is at the center of the Hall device (FIG. 7f); the center of the circle is between the centers of two Hall devices (FIGS. 7h and 7n); and the center of the circle is between the centers of four Hall devices (FIG. 7p). In the first case, in FIGS. 7a, 7c, 7i and 7k, the Hall device at the center of the circle has a voltage signal change of about 5 microvolts. From FIG. 7k, it is found that the two Hall sensors in the oblique direction have a signal change of about 2 microvolts. In the second case, in FIGS. 7b, 7e, 7g and 7j, the Hall device at the center of the circle has a signal change of about 3-4 microvolts. It is found from FIG. 7j that the other Hall device near it has a signal change of about 1 microvolt. In the third case, in FIGS. 7d, 7l, 7m and 7o, the two Hall devices close to the center of the circle have a signal change of about 2 microvolts, while the two Hall devices far from the center of the circle have a signal change of about 1 microvolt. In the fourth case, in FIG. 7f, the Hall device at the center of the circle has a signal change of about 5 microvolts, while the four Hall devices around it have a signal change of less than 1 microvolt, but greater than zero (0) microvolts. In the fifth case, in FIGS. 7h and 7n, the two nearby Hall devices have a signal variation of about 2 microvolts. In the sixth case, in FIG. 7p, the four nearby Hall devices have a signal variation of about 2 microvolts. At the same time, it can be seen that the signal output generated by 2-micron particles is greater than that generated by 1-micron particles. In theory, the larger the particles, the more magnetization they generate, and the greater the additional magnetic field they generate. In addition, when the Hall devices in the fifth row and fifth column near 1 micron and 2 micron are present, the voltage signal is significantly higher than when a single particle is present.

From FIGS. 7a-7p, it can be seen that the presence and position of particles can be inferred from the voltage changes of Hall devices in the array.

1. A dual-mode sensor configured to detect breast cancer cells in blood samples, comprising:
a metal on or near a surface of the sensor, which when electrified, generates a first magnetic field that attracts magnetic particles combined with, linked to or bound to breast cancer cells;
a magneto resistive sensor having a resistance affected or changed by a stray field from the magnetic particles, the magneto resistive sensor having a size equivalent or similar to that of the magnetic particles, and configured to detect the magnetic particles combined with, linked to or bound to the breast cancer cells;
a Hall magnetic sensor array, having a voltage affected or changed by the stray field from the magnetic particles and a size equivalent or similar to that of the magnetic particles, and configured to detect the magnetic particles combined with, linked to or bound to the breast cancer cells;
baffles on walls and a center of the magneto resistive sensor, the baffles forming a grid accommodating one of the magnetic particles per cell of the grid, configured to linearize a relationship between the resistance of the magneto resistive sensor and the number of the magnetic particles in the grid;
wherein the Hall magnetic sensor array is configured to sense a position of each of the magnetic particles on the surface of the sensor, and the magneto resistive sensor is configured to sense a number of the magnetic particles on the surface of the sensor.

2. The dual-mode sensor of claim 1, wherein the magneto resistive sensor comprises a tunnel magneto resistance (TMR) device.

3. The dual-mode sensor of claim 2, wherein the TMR device has a shape selected from rectangular, circular, and square.

4. The dual-mode sensor of claim 3, wherein the TMR device has the rectangular shape.

5. The dual-mode sensor of claim 1, wherein the magneto resistive sensor comprises an anisotropic magnetoresistive sensor (AMR) or a giant magnetoresistive sensor (GMR).

6. The dual-mode sensor of claim 1, wherein the metal comprises one or more wires configured to attract the magnetic particles.

7. The dual-mode sensor of claim 1, wherein the metal comprises a coil.

8. The dual-mode sensor of claim 7, wherein the coil comprises a Helmholtz coil.

9. The dual-mode sensor of claim 1, wherein the metal comprises a permanent magnet.

10. The dual-mode sensor of claim 1, wherein the cells of the grid have length, width and depth dimensions that accommodate a maximum of two of the magnetic particles.
